(19) United States
(12) Patent Application Publication
(10) Pub. No.: US 2026/0248591 A1
(43) Pub. Date: Aug. 27, 2026

(54) Medical and Dental Bioceramic Composition for Temporary Use

(71) Applicants: ANGELUS INDÚSTRIA DE PRODUTOS ODONTOLÓGICOS S/A, LONDRINA-PR (BR); ANGELUS JAPAN, OSAKA (JP)

(72) Inventors: CÉSAR EDUARDO BELLINATI, LONDRINA (BR); CARLA AKIMI KAWAGUTI, LONDRINA (BR); WILIAM PEREIRA DOS SANTOS, LONDRINA (BR); EDUARDO LIMA COSTA, LONDRINA (BR)

(21) Appl. No.: 19/117,997

(22) PCT Filed: Dec. 28, 2021
(86) PCT No.: PCT/BR2021/050576

§ 371 Date: Apr. 3, 2025

Related U.S. Application Data
(63) Continuation-in-part of application No. 17/562,146, filed on Dec. 27, 2021, now abandoned.
(60) Provisional application No. 63/131,411, filed on Dec. 29, 2020.

(51) Int. Cl. A61C 5/50

(52) U.S. Cl. CPC A61C 5/50

(57) ABSTRACT

The present invention is related to a bioceramic composition for temporary intracanal medication which promotes the constant, controlled and balanced release of metal ions, hydroxyl (OH—) and metal-salicylate complexes, being able of providing bioactive, antimicrobial and anti-inflammatory properties. The present invention further provides methods for preventing or controlling endodontic infection and for promoting tissue regeneration and repair by using said bioceramic composition.

### FIELD OF THE INVENTION

The present invention is related to cements useful in medical and dental applications, and more particularly to stable bioceramic compositions for temporary intracanal medication that are able to promote bioactivity.

### BACKGROUND OF THE INVENTION

Caries is a process caused by bacteria that leads to the destruction of dental tissues and can lead to the loss of the dental element if not treated in time. Once installed, its evolution can be divided into 3 phases. In the first stage, caries affects only the enamel; in a second phase it deepens and invades the dentin; in the third phase it reaches the root canal causing an endodontic infection. In the second and mainly in the third phase, toothache starts, caused mainly by the inflammatory process caused by bacterial aggression.

The need to prevent or control root canal infection, aiming the repair of the periradicular structures and the restoration of normal dental and oral health function, forms foundation on the solid which the contemporary endodontics rests on. Endodontic treatment has three main stages of infection control: chemical-mechanical preparation, intracanal medication and filling of the root canal system.

Although a considerable reduction in the number of bacterial cells in the main canal light can be obtained by chemical-mechanical effects of the instrumentation and irrigation, bacteria can remain viable in regions inaccessible to them. While minor anatomical irregularities can be incorporated into the preparation, areas such a recesses, isthmus, lateral and apical ramifications and dentinal tubules can harbor bacteria that, once not eliminated, put the result of the treatment at risk. These areas are not commonly affected by instruments and the auxiliary chemical substance, used for irrigation, will not have enough intracanal action time to act in depth and avoid infections.

In this sense, an intracanal medication having antibacterial activity is more likely to reach areas not affected by canal instrumentation, especially if it remains for a longer time inside the root canal. Thus, performing its antibacterial action, it can decisively contribute to the maximum reduction of the endodontic microbiota. By potentiating this reduction, the use of intracanal dressings is directly related to better repair of periradicular tissues.

### The Calcium Hydroxide

In dentistry, calcium hydroxide-based products have been used as an intracanal medication due to its ability to meet some properties that are desirable for medications of this nature, such as the promotion of healing and microbiological control of the conduit, for example. Among its main features, is its ability to increase the alkalinity of the conduit due to the calcium hydroxide dissociation mechanism into calcium and hydroxyl ions. These ions possess the ability to penetrate the dentinal tubules preventing the growth of new microorganisms, controlling and inhibiting possible infection, thus, imparting antimicrobial function to calcium hydroxide.

The problem of using calcium hydroxide-based intracanal medications is that the high rate of ion release, in a short time frame, may cause damage to surrounding tissues. Thus, the prolonged use of these calcium hydroxide-based products can compromise fracture resistance of the dentin structure. Another disadvantage of these products is the high solubility, being able to generate micro infiltration and, in some cases, reabsorption by vital tissues (periapical).

Another problem is that the calcium hydroxide paste when in contact with the pulp causes the formation of a layer of necrotic tissue that develops a calcified layer and can solubilize and stimulate the release of bioactive dentin molecules that stimulate calcified tissue formation [Yang S F, Rivera E M, Baumgardner K R, Walton R E, Stanford C. Anaerobic tissue-dissolving abilities of calcium hydroxide and sodium hypochlorite. J. Endod. 1995; 21:613-6]. Coagulation necrosis and the formation of the mineralized barrier implies on loss of vital tissue. This fact, in a way, can reduce the potential of pulp response in cases of subsequent pathologies. As disadvantages, it is pertinent to emphasize the possibility of inducing ectopic calcifications or the formation of an irregular or incomplete mineralized barrier. In addition, calcium hydroxide is not presented as a material able to promote bioactivity and its ability to completely eradicate the bacterial species in the root canals is questioned [Sathorn, C., Parashos, P. and Messer, H. (2007), Antibacterial efficacy of calcium hydroxide intracanal dressing: a systematic review and meta-analysis. International Endodontic Journal, 40:2-10)].

### Bioactive Properties

Bioceramics are an advantageous alternative to calcium hydroxide. They can be classified as bio-inert, bioactive or bioresorbable material based on their surface chemical reactivity [Heness G, Ben-Nissan B (2004). Innovative bioceramics. Materials Forum, 27, 104-14; Hench L L, Thompson I (2010) “Twenty-first century challenges for biomaterials.” Journal of the Royal Society Interface, 7, S379-S391]. Bioactive materials are materials able to form a chemical bond with living tissue. In the context of bone substitute material, the bioactivity of a material is regularly characterized by their ability to induce the formation of the apatite layer on its surface after being immersed into biological fluids [Hench L L, Splinter R J, Allen W, Greenlee T (2004) “Bonding mechanisms at the interface of ceramic prosthetic materials.” Journal of Biomedical Materials Research, 5, 117-141].

After a preliminary definition of the biomaterial in the 1950s, which was primarily based on the criteria of maximum biochemical and biological inertness in contact with body fluids (first generation of implantable materials), the discovery of the bioactive glass by Larry L. Hench in 1969 was the first inorganic material to present bioactivity and an alternative to the materials used in implants at the time.

One of the main features of the bioactivity of such bioactive glasses was based on the activity of Ca and Si ions present in their composition, that were able to induce the formation of a carbonated hydroxyapatite layer similar to the bone mineral phase on its surface [Baino F, Hamzehlou S, Kargozar S (2018) “Bioactive Glasses: Where Are We and Where Are We Going?” Journal of Functional Biomaterials 9, 25].

The second generation of bioactive glasses was able to promote a positive response from the living system by forming a strong and stable tissue-implant bond with the tissues where they were implanted, expanding the concept of biocompatibility [Fiume E, Barberi J, Verné E, Baino F (2018) “Bioactive Glasses: From Parent 45S5 Composition to Scaffold-Assisted Tissue-Healing Therapies.” Journal of Functional Biomaterials 9, 24].

In the '80s, it was found that the bioactive glasses were able to promote regeneration and stimulate osteogenesis and the bone formation process when they were used in particulate form. Later, it was discovered that metal ions present in the composition and released by the dissolution of bioactive glasses were responsible for the stimulation of growth and cell differentiation factors [Hench L L and Jones J R (2015) “Bioactive Glasses: Frontiers and Challenges.” Frontiers in Bioengineering and Biotechnology 3, 194]. One way found for the use of bioactive glasses is in the form of an “scaffold” which limits its clinical application [Wu C and Chang J (2013) “A review of bioactive silicate ceramics.” Biomedical Materials 8, 0320017.

In the late 60's, the interest in the use of various ceramic materials for biomedical applications arises as an alternative to the bioactive glasses, mainly because they present low solubility and improved mechanical strength. A little later, these materials were called Bioceramics [Dorozhkin S V (2010) “Calcium Orthophosphates as Bioceramics: State of the Art.” Journal of Functional Biomaterials, 1, 22-107].

Over the past few years, new dental compositions based on bioceramics have been proposed to replace calcium hydroxide and glass ionomer cements, with the main objective of increasing bioactivity. Among these new bioceramics are the classes of calcium silicate-based cements, which are ceramics that present themselves as an alternative source of calcium, available in the form of powder/liquid or in the form of pastes.

In the context of the use of bioactive materials in dentistry, Torabinejad proposed in U.S. Pat. No. 5,415,547 granted on May 16, 1995, a ceramic material for repairing dental structures based on Portland cement, that was able to release ions, recognized as MTA. Despite using calcium silicates as calcium ions source, which are less soluble than calcium hydroxide, the product has low physico-chemical properties as it is practically powdered Portland cement with the addition of a radiopacifier.

Although being considered as materials having some bioactivity and despite its degradation products do not cause an inflammatory reaction, calcium silicate-based cements however present numerous drawbacks in relation to their physical and biological properties, including: low mechanical resistance, making them unsuitable for load support application; and low chemical instability (high degradation rate) leading to a highly alkaline condition in the surrounding environment, which makes it detrimental to cell viability and limits its long-term biological use.

Studies show that there is a relationship between the rate of ion release and the bioactivity of the materials. Although compositions rich in calcium may seem more attractive for providing a faster release of Ca²⁺ ions and for ease the formation of the apatite hydroxide layer on its surface, calcium does not appear to be an essential element for the ceramics have bioactivity.

Yang described in U.S. Pat. No. 8,475,811 issued on Jul. 2, 2013, a formulation of hydraulic cement, in single and injectable paste, based on calcium silicate for use in dentistry and orthopedics. The focus of this invention was to obtain a pre-mixed paste with the presence of calcium silicates and a liquid carrier, with the ability to harden with the humidity of the physiological media. In the formulation developed by Yang, the setting mechanism occurs by hydrating the tricalcium silicate (Ca₃SiO₅) and dicalcium silicate (Ca₂SiO₄) phases that in contact with the humidity of the physiological medium hydrate and form two new phases: a calcium hydroxide (Ca(OH)₂) phase and a hydrated gel silicate phase (3CaO·2SiO₂·3H₂O) known as C—S—H. The entanglement of this C—S—H phase together with the calcium hydroxide (Ca(OH)₂) plates from the saturation of the medium decreases the mobility of the particles and promotes the setting of the material (hardening). However, the ready-to-use cement developed by Yang is not applied as a temporary intracanal medication, because its hardening occurs after the hydration process in the physiological environment, making it impossible to use it as a temporary material.

### Intracanal Medications

Conventional intracanal treatments have specific limitations and are sometimes restricted to irrigating fluids. For example, sodium hypochlorite and calcium hydroxide do not have the ability to eradicate all bacteria from the root canal system. Sodium hypochlorite and calcium hydroxide need to be in direct contact to be effective, but this is difficult to achieve. Direct contact cannot be obtained when calcifications, which are natural obstructions, are present. Furthermore, as described in U.S. Pat. No. 10,226,403 B2 issued on Mar. 12, 2019, when chlorhexidine is mixed with sodium hypochlorite during instrumentation, an orange-brown precipitate which is difficult to remove and can stain is formed.

Therefore, it is important to understand that the different means of antibacterial irrigation in root canal, by itself, are part of a concerted effort to control infections in endodontics. Alone, they cannot guarantee success if there are problems with the quality of some other parts of the treatment.

Accordingly, the intracanal medicament is defined as the temporary placement of medications having biocompatibility in the root canal, with the purpose of inhibiting coronary invading bacteria. To achieve this goal, the ideal intracanal medication must be an effective antimicrobial agent with a long-term effect. Other desirable features include, without limitation, not being irritating to periradicular tissues, so as not to interfere with its repair and be active in the presence of blood, serum, and tissue protein derivatives.

Although one of the main goals of intracanal medication is to inhibit microbial growth for a certain period, the compositions based on calcium hydroxide currently available on the market, are not able to significantly stimulate tissue regeneration and repair during treatment.

U.S. Pat. No. 4,240,832 A, issued on Dec. 23, 1980, discloses a composition based on calcium hydroxide, a condensate of an salicylic acid ester and pulp capping aldehydes.

U.S. patent application 2013/0023601 A1, published on Jan. 24, 2013, reports a cementitious composition based on calcium silicate and salicylic acid esters with indication for intermediate restorations and canal filling dental procedures.

Both documents differ from the present invention in that the compositions are proposed in a two-paste system and that, when applied, have the feature of hardening at the site of application. Therefore, in addition to including the need for a homogenization step between two different pastes, that may cause problems related to the process of application of the material, both compositions have the feature of hardening during use, which prevent them from being applied as a temporary intracanal medication.

Patent application EP 2 736 519 A1, published on Jan. 31, 2013, reports an alkaline composition with antimicrobial activity comprising calcium hydroxide and a triol compound. U.S. patent application 2014/0234442 A1, published on Aug. 21, 2014, reports a composition for filling dental root canals comprising a linezolid antibiotic agent, calcium hydroxide and other pharmaceutically acceptable excipients.

Patent application WO 2011/102724 A2, published on Aug. 25, 2011, reports a composition for the antimicrobial treatment of dental root canals achieved by combining calcium hydroxide with potassium iodide and chlorhexidine to promote antimicrobial activity.

These documents differ from the present invention because they disclose compositions based on the fact of using calcium hydroxide as a source, to confer its antimicrobial properties. In addition, none of the compositions proposed by these documents have any regeneration bioactive properties when they are applied.

Therefore, there is a need to develop intracanal medications that also have bioactive properties through the controlled and stable release of metal ions, providing better biological responses, in addition to possessing broad spectrum antimicrobial, anti-inflammatory and physicochemical properties suitable for handling and easy removal after use. To achieve this goal it is necessary to use chemical elements whose electronic distribution allows the formation of metal ions in specific crystalline arrangements and thus stimulates a desired biological response.

Thus, the technical problem to be solved by the present invention is related to providing a biomaterial that, besides having an antimicrobial and anti-inflammatory character, allows its easy manipulation and is able to provide metal ions having bioactive properties without setting, allowing its use as a temporary intracanal medication.

### SUMMARY OF THE INVENTION

In view of the foregoing, exists in the art, a deficiency of a intracanal medication that show suitable bioactivity properties and physical-chemical properties proper for use as temporary material. To meet these requirements, the present invention uses a controlled source of metal ions in a constant and balanced way, which allows the induction of cell differentiation and, in this way, increases the capacity for repair and regeneration events of tissue-bone and dentin-pulp complexes.

Thus, the object of the present invention is to provide a temporary intracanal medication that present antimicrobial and anti-inflammatory properties and which, when in contact with the physiologic environment, promotes the constant, controlled and balanced release of metal ions (M₁^{+x}), hydroxyl (HO⁻) and metal salicylate cationic complex, thus providing a bioactive effect and having antimicrobial and anti-inflammatory properties.

The present invention provides a bioceramic composition for temporary intracanal medicament comprising at least one complexing resin and a metal ions source. Said composition upon contact with a saline solution forms a cationic complex having metal ions-controlled release, thus, imparting bioactive, antimicrobial and anti-inflammatory properties to the composition.

The present invention further provides methods for preventing or controlling endodontic infection and for promoting tissue regeneration and repair by applying the bioceramic composition of the present invention to the root canal of an subject in need thereof.

### BRIEF DESCRIPTION OF THE FIGURES

FIG. 1 shows the results of the pH assay carried out for the bioceramic compositions of the present invention.

FIG. 2 illustrates the evaluation of sections stained with hematoxylin and eosin (HE) for each group for counting the number of inflammatory cells.

FIG. 3 shows the results for counting the number of inflammatory cells in the groups for the different days analyzed.

FIG. 4 illustrates the evaluation of the groups after induction of immunohistochemical reactions for detection of interleukin-6 (IL-6) and interleukin-10 (IL-10).

FIG. 5 shows the results for counting the number of interleukin-6 (IL-6) in the groups for the different days analyzed.

FIG. 6 shows the results for counting the number of interleukin-10 (IL-10) in the groups for the different days analyzed.

FIG. 7 illustrates the evaluation of the groups for the presence of collagens.

FIG. 8 shows the results of the presence of collagen in the groups for the different days analyzed.

FIG. 9 illustrates the results of the von Kossa method carried out for detecting calcium and phosphate deposits.

FIG. 10 illustrates the results of the survival curve of E. faecalis against bioceramic composition (TP3), Ca hydroxide paste (CHP) and standard Chlorhexidine (CLX).

### DETAILED DESCRIPTION OF THE INVENTION

Although the present invention can be presented in different embodiments, the present specification along with the drawings, indicate a preferential embodiment, emphasizing that it should be considered an example of the core of the invention and not a limitation.

The present invention is related to a bioceramic composition for temporary intracanal medication comprising at least one cation complexing resin derived from salicylic acid ester and a source of metal ions. The composition of the present invention has bioactive, anti-inflammatory and antimicrobial properties and is indicated for temporary intracanal medications for dental treatment.

In an preferred embodiment, the bioceramic composition for temporary intracanal medication of the present invention is composed by a paste which may or may not be ready for use comprising, essentially, one source of metal ions and at least one salicylic acid ester derivative.

The source of metal ions present in the bioceramic composition for intracanal medication of the present invention, when in contact with body fluid, release metal ions (M⁺=Ca⁺², Mg⁺², Sr⁺², Zn⁺², Zr⁺⁴) and hydroxyl ions (OH) through the break of Si—O-M bonds. The reaction is shown below:

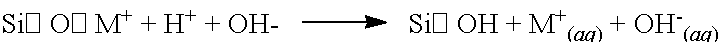

Further, the hydroxyl ions (OH⁻) remove the hydrogen from the salicylic acid ester group structure (R—C₇H₄O₂—OH) resulting in a molecule of water and one salicylate ion (R—C₇H₄O₂—O)⁻. Next, a complexing reaction between the salicylate ion and the metal ion (M^{+x}) with the formation of one metal salicylate complex is carried out, as described in the equation below:

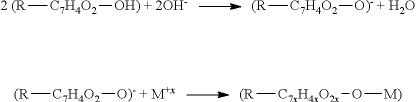

In which, R is a group selected from methyl, ethyl, n-butyl, isobutyl, propyl, hexyl, benzyl and diester; and X represents the valence of the metal ion.

Depending on the X valence, the metal-salicylate complex forms different structures, and may have one to four salicylate groups attached to the metal.

Depending on the group R, the metal-Salicylate complex undergoes different rates of dissociation, supplying metal ions to the medium in a modulated manner, following the equation below:

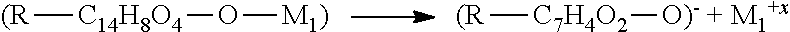

This process occurs continuously as the source of metal ions undergoes hydration by the physiological environment.

Therefore, the metal dissociation of the salicylate complex is carried out for a long period of time giving the bioceramic composition potential application as an intracanal medication, since it provides for a sufficient exposure time allowing the development of bioactivity.

In order to maintain this modulating effect of the proposed bioceramic composition, it is necessary that the ratio of complexation resin/metal ions source is lower than 1:3. The correct proportion of metal ions and complexing resin in the same composition allows the formation of metal-salicylate complexes without the phenomenon of setting/hardening of the composition, which therefore allows easy removal of the composition after it has been maintained in the root canal during the treatment.

Furthermore, the metal-salicylate complex formed shows anti-inflammatory properties due to its activity in free radicals. Free radicals are highly reactive species generated in living organisms for the protecting purpose. However, in some circumstances, they are responsible for the tissue damage or aggravation thereof. Metal-salicylate complex has direct activity on free radicals and non-radicular reactive species, which contributes to its activity against inflammation. The metal complexation of different salicylates has been a strategy used for improving pharmacological activity of different molecules and for reducing their side effects.

After the total formation of the metal-salicylate complex, the consumption of ions M₁^{+x} and OH ceases. The increase in the concentration of hydroxyl ions (OH) in physiological medium promotes the increasing of the pH of the solution to values greater than 10, turning the medium alkaline and unsuitable for microorganisms growth, thus, giving antimicrobial properties to the bioceramic composition for intracanal medication.

In addition, the steady concentration of multiple metal ions in the media, enables enzymatic changes which influence and stimulate tissue formation, promoting repair and regeneration of the affected area and, then, giving bioactive properties to the bioceramic composition for intracanal medication.

In a preferred embodiment of the present invention, the bioceramic compositions for temporary intracanal medication will be made available in the form of a single paste, also comprising an inert liquid carrier. Suitable and non-limiting examples of inert liquid carrier are materials derived from the glycol group, for example, ethylene glycol; propylene glycol; polyethylene glycol; polypropylene glycol; glycerin; diethylene glycol dimethyl ether; diethylene glycol monoethyl ether; butylene glycol, or the combination thereof.

The metal ions source used in the materials of the present invention are able to release metal ions from Ca, Mg, Sr, Zn, Zr or a combination of them. Suitable but not limiting examples, are selected from the group of metal silicates and aluminates, preferably from the group of metal silicates.

In preferred embodiments, metallic silicates are selected from Alite (3CaO·SiO₂), Belite (2CaO·SiO₂), Strontium-akermanite (Sr₂MgSi₂O₇), Akermanite (Ca₂MgSi₂O₇), Baghdadite (Ca₃ZrSi₂O₉), Hardistonite (Ca₂ZnSi₂O₇) or combinations thereof.

Suitable complexing resin belongs to the group of compositions of salicylic acid ester derivative. Nonlimiting examples are selected from the group consisting of methyl salicylate, ethyl salicylate, n-butyl salicylate, isobutyl salicylate, propyl salicylate, hexyl salicylate, benzyl salicylate and diester or combinations thereof.

In a preferred embodiment, the salicylic acid ester is methyl salicylate or diester.

An feature of the composition important for its use as a temporary intracanal medication is radiopacity, that is, the ability of the composition to block the X-rays used in a radiological examination. To impart this property to the composition of the present invention, several radiopacifying agents can be used, for example, but not limited to, barium, bismuth, rare earth derivatives, strontium, zirconium, silicon, aluminum, titanium, tungsten, among other radiopacifying agents.

Suitable radiopacifying agents are barium sulfate, zirconium oxide, bismuth oxide, tantalum oxide, titanium oxide and calcium tungstate, or a combination thereof.

In a preferred embodiment, the bioceramic composition of the present invention comprises at least:
i) 1 to 10% by weight of a complexing resin derived from salicylic acid ester;
ii) 10 to 60% by weight of a liquid carrier;
iii) 20 to 60% by weight of a radiopacifying agent; and
iv) 3 to 30% by weight of a metal ion source.

In a most preferred embodiment, the bioceramic composition of the present invention comprises at least:
i) 5 to 7% by weight of a complexing resin derived from salicylic acid ester;
ii) 42 to 44% by weight of a liquid carrier;
iii) 25 to 27% by weight of a radiopacifying agent; and
iv) 20 to 22% by weight of a metal ions source.

In another embodiment, the present invention provides a method for preventing or controlling endodontic infection by applying the bioceramic composition of the present invention in the root canal of a subject in need thereof.

In a further embodiment, the present invention provides a method for promoting tissue regeneration and repair by applying the bioceramic composition of the present invention in the root canal of a subject in need thereof.

In a further embodiment, the present invention provides for the use of the bioceramic composition of the present invention for preparing a product for preventing or controlling endodontic infection in a subject in need thereof.

In a further embodiment, the present invention provides for the use of the bioceramic composition of the present invention for preparing a product for promoting tissue regeneration and repair in a subject in need thereof.

To allow a better understanding of the present invention and to clearly demonstrate the obtained technical advances, the results of different tests carried out with respect to a non-limiting example of the invention are now presented.

### EXAMPLE

The bioceramic composition for temporary intracanal medication of the present invention (Table 1) are prepared by mixing the liquid carrier component and the complexing resin with a mechanical stirrer, and then adding the solid components: the metal silicate (metal ion source) and the radiopacifying agent with speed of less than 500 rpm, for approximately 45 minutes, until complete homogenization.

TABLE 1
Examples of the Bioceramic Compositions.

| | Metal ion | Radiopacifier | Liquid | Complexing |
|---|---|---|---|---|
| Sample | source | agent | carrier | resin |
| TP1 | Akermanite | Calcium | Polyethylene | Methyl |
| | (Ca₂MgSi₂O₇) | tungstate | glycol | salicylate |
| | 20% | 30% | 45% | 5% |
| TP2 | Baghdadite | Zirconium | Polyethylene | Diester |
| | (Ca₂ZrSi₂O₇) | oxide | glycol | 5% |
| | 20% | 30% | 45% | |
| TP3 | Alite | Calcium | Polyethylene | Methyl |
| | (Ca₃SiO₅) | tungstate | glycol | salicylate |
| | 20% | 30% | 45% | 5% |
| TP4 | Belite | Zirconium | Polyethylene | Diester |
| | (Ca₂SiO₄) | oxide | glycol | 5% |
| | 20% | 30% | 45% | |

pH, ion release, anti-inflammatory, bioactivity and antimicrobial assays were conducted with the compositions prepared in the Example.

### pH Assay

For the pH assay a small amount of the compositions were added into Eppendorf flasks and carried out in triplicate. Then, 1 mL of distilled and deionized water was added. The pH assay was carried out with a 900 μL aliquot of the supernatant retrieved after centrifugation at 1, 400 RPM for 3 minutes. After each assay, in 24 h (1 day), 3, 5, 10, 20 and 30 days, 900 μL of distilled and deionized water was added into each sample to enable the ion exchange.

### Metal Ion Release Assay

For the metal ion release assay, a small amount of the compositions were added into Eppendorf flasks and carried out in triplicate. Then, 1.5 mL of distilled and deionized water was added. The metal ion release assay was carried out with a 1,000 μL aliquot of the supernatant retrieved after centrifugation at 10,000 rpm for 3 minutes. The 1,000 μL aliquot was then transferred to a 5 ml beaker and diluted to 2 mL with distilled water. The assay was performed using the pH meter with a properly calibrated calcium electrode. After each assay, in 24 h (1 day), 3, 5, 10, 20 and 30 days, 1,000 μL of distilled and deionized water was added into each sample to enable the ion exchange.

Table 2, below, shows the results obtained in the pH and metal ions release assays. The results show that the compositions presented pH≈10 that is sufficient for antimicrobial activity and constant release of metal ions during the tested period of 30 days.

TABLE 2
Results of the physical-chemical assays of the bioceramic compositions.

| | Ionic concentration (ppm) | | | | | | | | pH | | | |
|---|---|---|---|---|---|---|---|---|---|---|---|---|
| Days | TP 1 | | TP 2 | | TP 3 | | TP 4 | | TP 1 | TP 2 | TP 3 | TP 4 |
| 1 | Ca | (182) | Ca | (112) | Ca | (223) | Ca | (201) | 10.9 | 10.5 | 11.9 | 11.6 |
| | Mg | (64) | Zr | (32) | Si | (35) | Si | (43) | | | | |
| | Si | (58) | Si | (45) | | | | | | | | |
| 3 | Ca | (180) | Ca | (109) | Ca | (121) | Ca | (198) | 10.5 | 9.9 | 11.6 | 11.2 |
| | Mg | (61) | Zr | (36) | Si | (33) | Si | (42) | | | | |
| | Si | (56) | Si | (44) | | | | | | | | |
| 5 | Ca | (177) | Ca | (108) | Ca | (120) | Ca | (195) | 10.5 | 9.8 | 11.5 | 11 |
| | Mg | (60) | Zr | (30) | Si | (32) | Si | (40) | | | | |
| | Si | (54) | Si | (42) | | | | | | | | |
| 10 | Ca | (176) | Ca | (107) | Ca | (118) | Ca | (194) | 10.6 | 9.9 | 11.4 | 11.2 |
| | Mg | (59) | Zr | (28) | Si | (29) | Si | (39) | | | | |
| | Si | (53) | Si | (39) | | | | | | | | |
| 20 | Ca | (173) | Ca | (101) | Ca | (115) | Ca | (189) | 10.2 | 9.6 | 11.2 | 10.9 |
| | Mg | (42) | Zr | (27) | Si | (25) | Si | (35) | | | | |
| | Si | (51) | Si | (32) | | | | | | | | |
| 30 | Ca | (171) | Ca | (100) | Ca | (111) | Ca | (184) | 10 | 9.4 | 11.2 | 10.6 |
| | Mg | (57) | Zr | (27) | Si | (23) | Si | (29) | | | | |
| | Si | (49) | Si | (31) | | | | | | | | |

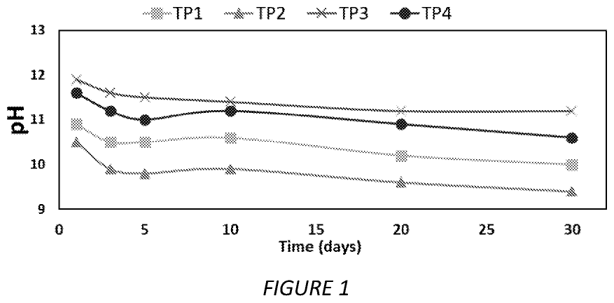

Furthermore, the results of the pH assay are illustrated in FIG. 1.

### Anti-Inflammatory and Bioactive Potential Assay

For the anti-inflammatory and bioactive potential assay, polyethylene tubes were implanted in the dorsal subcutaneous tissue of 60 rats, divided in groups of: bioceramic composition (TP3), Ca hydroxide paste (CHP) and control (empty CG-tubes). After 7, 15, 30 and 60 days, the animals were anesthetized and blood was collected by cardiac puncture to obtain serum for analysis. Subsequently, the animals were sacrificed and the implants with the adjacent tissues were removed and fixed in formaldehyde. Longitudinal sections were stained with Hematoxylin and Eosin (HE) for morphological analysis, and to obtain the number of inflammatory cells. Immunohistochemical reactions were induced for detecting interleukin-6 (IL-6) and interleukin-10 (IL-10). The bioactive potential was evaluated by the von Kossa method and by the analysis of non-colored sections under the microscope with polarized light, which were carried out to detect calcium and calcite crystals deposits, respectively. Some sections, after the von Kossa reaction, were subjected to immunohistochemical reaction to detect alkaline phosphatase, an enzyme produced by mineralized tissues cells.

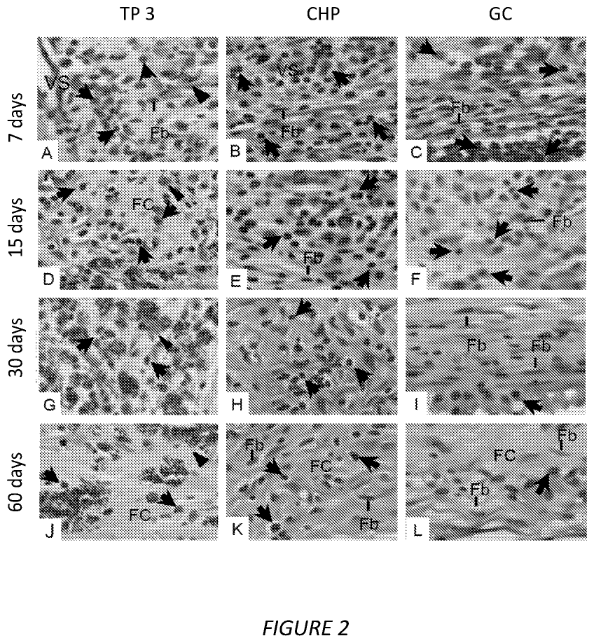

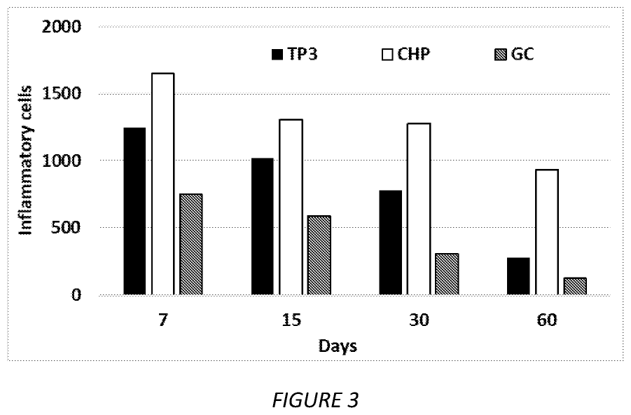

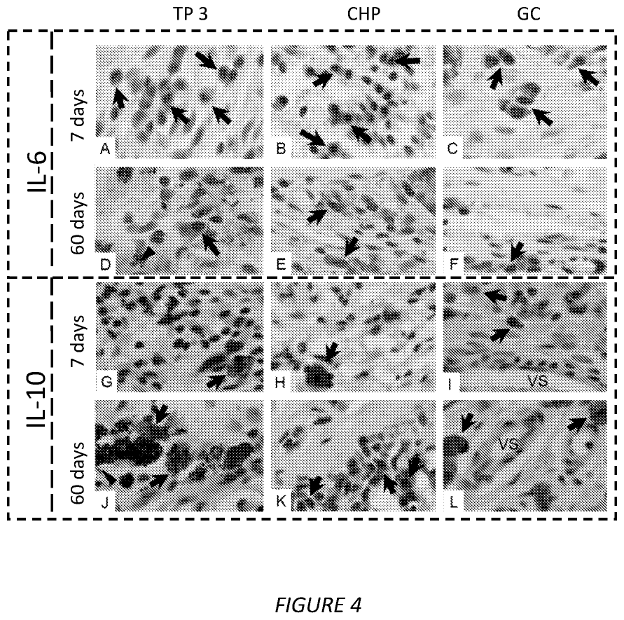

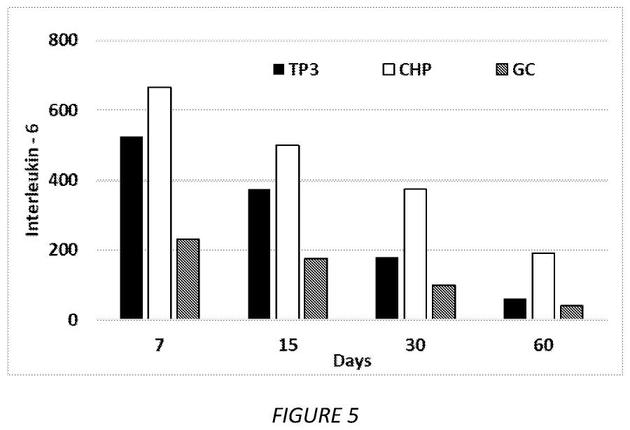

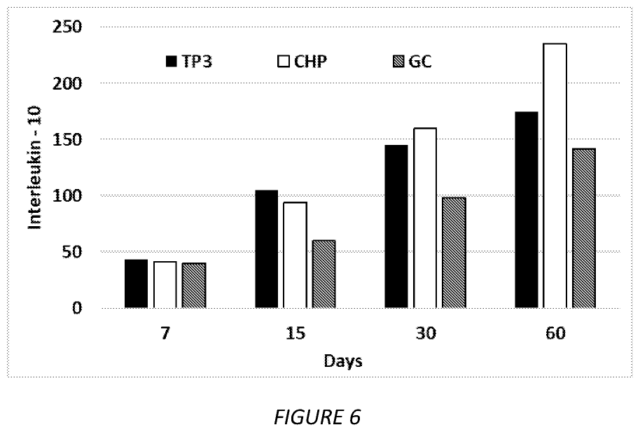

FIGS. 2, 3, 4, 5 and 6 show images of the results obtained for the measurement of the anti-inflammatory potential. The obtained results show numerical density of inflammatory cells and cells immunostained for IL-6 and IL-10. Significant differences in the number of IL-6 and IL-10 cells were seen between TP3 and CHP at 7, 15, 30 and 60 days. The significant decrease of the inflammatory process concomitant to the decrease of the immunoreactivity for IL-6 and IL-10 (FIGS. 5 and 6) indicates that proposed composition TP3 has significant anti-inflammatory features.

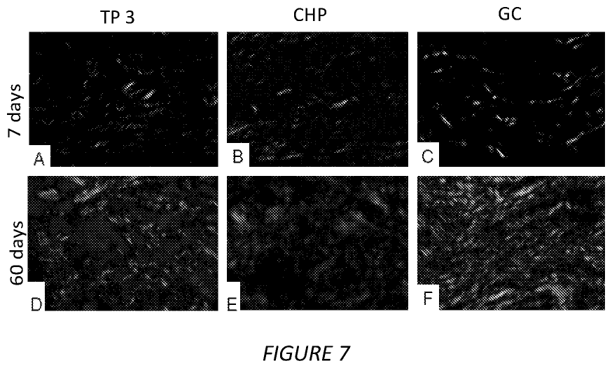

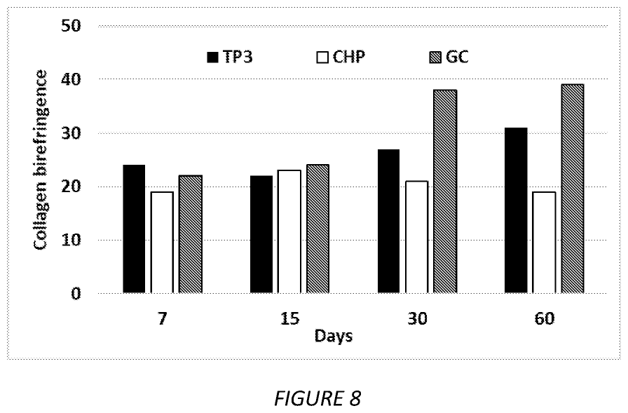

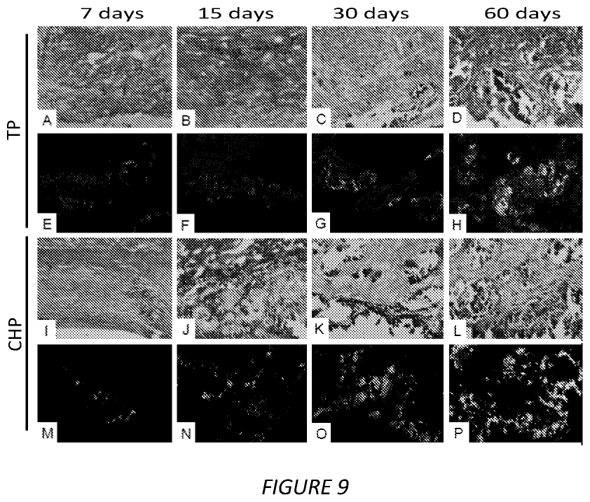

FIGS. 7, 8, and 9 show the results obtained for measuring the bioactive potential by the von Kossa method. It is possible to see a significant increase in the formation of calcium nodules in FIGS. 9C and 9D, as well as a significant increase in the formation of phosphate nodules in FIGS. 9G and 9H for TP3 composition, indicating that the composition of the present invention was able to increase osteoblasts mineralizing capacity. The presence of von Kossa positive structures and of birefringent structures suggests that the TP3 composition has expressive bioactive potential.

### Antimicrobial Assay

For the antimicrobial assay, each of a 150 mg of bioceramic composition (TP3), Ca hydroxide paste (CHP) and standard Chlorhexidine (CLX) was manipulated, in a Falcon tube, and 5 ml of ultrapure water was added and stirred in vortex for 1 min. Then, 2.8 mL of each mixture was taken and mixed in 100 μL aliquots of E. faecalis suspensions (3×107 CFU/mL), and in diluted chlorhexidine solution. The E. faecalis suspension was prepared with sterile 0.85% saline solution, equivalent to the MacFarland 1 scale (3×108 CFU/mL) and measured on a MacFarland densitometer (DEN-1). The suspension was stirred in vortex for 1 minute. After 1, 3, 6, 15 and 24 hours, 100 μL aliquots were taken from the samples. These samples were first transferred to a second tube containing a neutralizing agent and undergone to the decimal serial dilution from 10-1 to 10-5. The neutralizing agents used were 0.5% (w/v) acid citric for TPX and CHP solutions, and 0.3% (w/v) soy lecithin and polysorbate solutions for chlorhexidine. Aliquots of 100 μL of each of the dilutions were seeded in triplicate on the surface of Tryptic Soy Agar (TSa) plates and were incubated at 37° C. for 24 to 48 hours. The number of CFU/g (colony-forming units per gram) were counted using a colony counter and all the CFU values were converted to Log₁₀¹⁰.

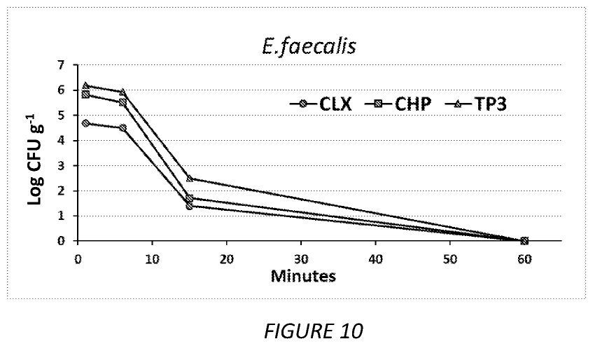

FIG. 10 shows the results of the survival curve of E. faecalis against bioceramic composition (TP3), Ca hydroxide paste (CHP) and standard Chlorhexidine (CLX). It is possible to see that the bioceramic composition (TP3) presented greater antimicrobial activity against E. faecalis than Ca hydroxide paste (CHP) and standard Chlorhexidine (CLX).

Although only some embodiments of the present invention have been shown, it will be understood that omissions, substitutions and changes may be made by a person of skill in the art without deviating from the spirit and scope of the invention.

Furthermore, it is expressly provided that the content of the documents mentioned in this specification is incorporated herein by reference.

1. A bioceramic composition for temporary intracanal medication characterized in that it comprises at least one complexing resin derived from salicylic acid ester and a metal ions source, wherein the complexing resin is present in from 1 to 10% by weight, and in which said composition promotes the formation of cationic complexes with controlled release of metal ions upon being in contact with a physiological solution.

2. The bioceramic composition, according to claim 1, characterized in that it also comprises a liquid carrier.

3. The bioceramic composition, according to claim 1, characterized in that it also comprises a radiopacifying agent.

4. The bioceramic composition, according to claim 1, characterized in that it comprises at least:
i) 10 to 60% by weight of a liquid carrier;
ii) 20 to 60% by weight of a radiopacifying agent; and
iii) 3 to 30% by weight of a metal ions source.

5. The bioceramic composition, according to claim 1, characterized in that it comprises at least:
i) 5 to 7% by weight of a complexing resin derived from salicylic acid ester;
ii) 42 to 44% by weight of a liquid carrier;
iii) 25 to 27% by weight of a radiopacifying agent; and
iv) 20 to 22% by weight of a metal ions source.

6. The bioceramic composition, according to claim 1, characterized in that the salicylic acid ester derivatives are selected from the following group: methyl salicylate, ethyl salicylate, n-butyl salicylate, isobutyl salicylate, propyl salicylate, hexyl salicylate, benzyl salicylate and diester or a combination thereof.

7. The bioceramic composition, according to claim 2, characterized in that the liquid carrier is selected from ethylene glycol; propylene glycol; polyethylene glycol; polyethylene glycol; glycerin; diethylene glycol dimethyl ether; diethylene glycol monoethyl ether; butylene glycol or a combination thereof.

8. The bioceramic composition, according to claim 3, characterized in that the radiopacifier agent is selected from the following group: barium sulfate, zirconium oxide, bismuth oxide, tantalum oxide, titanium oxide and calcium tungstate or a combination thereof.

9. The bioceramic composition, according to claim 1, characterized in that the metal ions source comprises at least one silicate containing metal ions selected from Ca, Mg, Sr, Zn, Zr or a combination thereof.

10. The bioceramic composition, according to claim 1, characterized in that the metal ions source is selected from the group consisting of Alite (3CaO·SiO₂), Belite (2CaO·SiO₂), Strontium-akermanite (Sr₂MgSi₂O₇), Akermanite (Ca₂MgSi₂O₇), Baghdadite (Ca₃ZrSi₂O₉), Hardistonite (Ca₂ZnSi₂O₇) or a combination thereof.

11. The bioceramic composition, according to claim 76, characterized in that the complexing resin is a salicylic acid ester derivative, wherein said salicylic acid ester is methyl salicylate or diester.

12. A method for preventing or controlling endodontic infection characterized in that it comprises applying the bioceramic composition as defined in claim 1 in the root canal of a subject in need thereof.

13. A method for promoting tissue regeneration and repair characterized in that it comprises applying the bioceramic composition as defined in claim 1 in the root canal of a subject in need thereof.
